(19) United States
(12) Patent Application Publication
(10) Pub. No.: US 2026/0168929 A1
(43) Pub. Date: Jun. 18, 2026

(54) SENSOR ASSEMBLY

(71) Applicant: WEAR DETECTION TECHNOLOGIES PTY LTD, Canning Vale (AU)

(72) Inventors: David Adam Bahemia, East Fremantle (AU); Daniel Berti, Duncraig (AU); Aaron Leech, Tamborine (AU); Ayrton Sue, Karrinyup (AU)

(21) Appl. No.: 18/737,302

(22) PCT Filed: Dec. 6, 2022
(86) PCT No.: PCT/AU2022/051460

§ 371 Date: Jun. 7, 2024

(30) Foreign Application Priority Data
Dec. 8, 2021 (AU) 2021903977

(51) Int. Cl. G01N 21/64; E21B 47/00; F17D 5/00; G01N 21/85; G01N 33/28

(52) U.S. Cl. CPC G01N 21/6428; E21B 47/00; F17D 5/00; G01N 21/85; G01N 33/2823; G01N 2021/6439; G01N 2201/062

(57) ABSTRACT

A sensor assembly for detecting a fluorophore in a well fluid includes a first light source for emitting a first light to excite the fluorophore at a predetermined point in the well fluid, a first light detector for detecting a light emitted from the fluorophore in response to the first light, and a light transparent member located between the well fluid and the first light source and first light detector and defining an outer surface and an inner surface in fluid communication with the well fluid.

### CROSS-REFERENCE TO RELATED APPLICATION

This application is a continuation of and claims priority under 35 U.S.C. § 363, to PCT/AU 2022/051460, filed Dec. 6, 2022, and to Australian patent application 2021/903,977, filed Dec. 8, 2021, the entireties of each are incorporated herein by reference.

### FIELD OF THE INVENTION

This invention relates to a sensor assembly for detecting a fluorophore in a well fluid. This invention also relates to a sensor unit including the sensor assembly and to a method for detecting wear in a well component.

### BACKGROUND OF THE INVENTION

Oil and gas wells use subsurface equipment for the extraction of the well fluid. In general, the downhole components either reciprocate or rotate to pump the well fluid to the surface. The key parts of the well are the well production tubing through which the well fluid from the well passes, the rod driving the action to pump the well fluid, and guides for separating the rod from the well production tubing and/or to enhance flow of the well fluid.

The rods, with attached guides, are assembled into a string that is then located in the well production tubing. During operation, contact between the components and/or with the well fluid results in wear. Excessive wear of the components can lead to component failure and/or render a well non-functional or to operate at sub-optimal levels. Therefore, components of the string, such as the guides, need to be changed out to continue efficient operation.

Decisions on when to change out a component can have an important impact on the efficient operation of a well and accurate data on component wear is therefore valuable as it informs and improves any such decisions.

The present invention seeks to provide a sensor assembly, sensor unit and method that addresses or at least partially ameliorate the problems with existing wear detection systems. At the very least, the present invention seeks to provide a useful alternative to currently available solutions.

Therefore, a need exists to overcome the problems with the prior art as discussed above.

### SUMMARY OF THE INVENTION

According to a first aspect, the present invention provides a sensor assembly for detecting a fluorophore in a well fluid, the sensor assembly including:
(a) a first light source for emitting a first light to excite the fluorophore at a predetermined point in the well fluid;
(b) a first light detector for detecting a light emitted from the fluorophore in response to the first light; and
(c) a light transparent member located between the well fluid and the first light source and first light detector and defining an outer surface and an inner surface in fluid communication with the well fluid.

According to a second aspect, the present invention provides a sensor unit that includes a sensor assembly according to a first aspect of the present invention; and a signal processor for processing a signal generated by the sensor assembly.

According to a third aspect, the present invention provides an insert for a pipeline including a sensor assembly according to a first aspect of the present invention or a sensor unit according to a second aspect of the present invention; and a well fluid conduit defining an inlet and an outlet.

According to a fourth aspect of the invention, the present invention provides a method of detecting wear of a well component in a well, the method includes the steps of:
(a) incorporating a well component, including a fluorophore, in the well; and
(b) assaying the well fluid for the fluorophore;
   wherein the presence of the fluorophore in the well fluid is indicative of the wear.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1a is perspective view from the front and below of an insert for a pipeline according to an embodiment of the third aspect of the present invention;

FIG. 1b is front view of the insert for a pipeline in FIG. 1a;

FIG. 1c is view through cross section A-A in FIG. 1b;

FIG. 1 d is view of detail B from FIG. 1 c;

FIG. 1e is bottom view of the insert for a pipeline in FIG. 1a;

FIG. 1f is view through cross section O-O in FIG. 1e;

FIG. 1 g is view of detail D from FIG. 1 f;

FIG. 1h is a diagram of a well including the insert of FIG. 1a and other well components;

FIG. 2 is a schematic diagram of a sensor assembly according to one embodiment of the first aspect of the present invention; and

FIG. 3a is a perspective view of a part of a sensor assembly (omitting the light transparent member) according to another embodiment of the first aspect of the present invention;

FIG. 3b is a side view of the sensor assembly in FIG. 3a from direction X;

FIG. 3c is view through cross section G-G in FIG. 3b;

FIG. 3 d is view of detail E from FIG. 3 c;

FIG. 4 a is an end view of the part of the sensor assembly in FIG. 3 a from direction Z and shows the LED side of the sensor assembly “1” and the photodiode side of the sensor assembly “2”;

FIG. 4b is a perspective view from below of the part of the sensor assembly in FIG. 4a;

FIG. 4c is a perspective view from below of the part of the sensor assembly in FIG. 4a from the opposite side;

FIG. 4d is view through cross section I-I in FIG. 4a;

FIG. 4e is view through cross section J-J in FIG. 4a; and

FIG. 5 is a perspective view from the front and above of an insert for a pipeline according to a second embodiment of the third aspect of the present invention.

### DETAILED DESCRIPTION

According to a first aspect, the present invention provides a sensor assembly for detecting a fluorophore in a well fluid, the sensor assembly including:
(a) a first light source for emitting a first light to excite the fluorophore at a predetermined point in the well fluid;
(b) a first light detector for detecting a light emitted from the fluorophore in response to the first light; and
(c) a light transparent member located between the well fluid and the first light source and first light detector and defining an outer surface and an inner surface in fluid communication with the well fluid.

The fluorophore may have an excitation wavelength of 325-425, 345-400, 350-390, 360-380, 365-375, 365, 366, 367, 368, 369, 370, 371, 372, 373 or 374 nm and may be a dye. The fluorophore may be 1,3,6,8-pyrene tetrasulfonic acid tetrasodium salt (PTSA) and may also be rhodamine, fluorescein, eosin, or dansyl.

The well fluid may have a turbidity of at least 1000 NTU, 2000 NTU, 3000 NTU, 4000 NTU, 5000 NTU, 7500 NTU, 10000 NTU, 12500 NTU, 15000 NTU, 17500 NTU, 20000 NTU, 22500 NTU, 25000 NTU, 27500 NTU, 30000 NTU. The well fluid may be a slurry and may include a particulate material, such as rock, clay, and/or sand. The well fluid may be oil and/or gas.

The first light source may be a light emitting diode (LED). The first light source can have a leading end facing the light transparent member and a trailing end facing the opposite direction. The first light source can be oriented such that, when in use, its central axis defines an acute angle with the outer surface of the light transparent member. The acute angle can be about 40-80, 50-70, 55-65 or 60°. The first light source can further include a first filter that only allows the passage of light of a predetermined wavelength or wavelength range.

The predetermined wavelength or wavelength range includes a wavelength that is adapted to excite the fluorophore. The predetermined wavelength or wavelength range can include 320-380, 325-375, 335-365, 345-355, 348, 349, 350, 351 or 352 nm.

The first light may be of a wavelength or wavelength range that is adapted to excite the fluorophore, such as 325-425, 345-400, 350-390, 360-380, 365-375, 365, 366, 367, 368, 369, 370, 371, 372, 373 or 374 nm.

The first light detector may be a photodiode, a phototransistor, or a photoresistor. It may include a leading end facing towards the light transparent member and a trailing end facing in the opposite direction. The first light detector can be oriented such that, when in use, its central axis defines an acute angle with the outer surface of the light transparent member. The acute angle can be about 40-80, 50-70, 55-65 or 60°. The first light detector can be oriented such that its central axis and the central axis of the first light source converge in a direction towards the predetermined point to define an angle of convergence. The angle of convergence can be an acute angle, for example, about 40-80, 50-70, 55-65 or 60°.

The first light detector further includes a second filter that only allows the passage of light of a predetermined wavelength or wavelength range. The predetermined wavelength or wavelength range can be a wavelength or wavelength range including the emission wavelength of the fluorophore. For example, the predetermined wavelength or wavelength range can be 390-460, 400-450, 410-440, 420-430, 421, 422, 423, 424, 425, 426, 427, 428 or 429 nm. The first light detector can be located in a position opposed to the first light source, such that the first light source and the first light detector are opposed to each other.

The light emitted from the fluorophore can include a wavelength or wavelength range of 390-460, 400-450, 410-440, 420-430, 421, 422, 423, 424, 425, 426, 427, 428 or 429 nm.

The first light source and the first light detector can form a first optical arrangement for detecting the fluorophore.

The predetermined point can be located proximal to the inner surface of the light transparent member. The predetermined point can be equal to or less than 20, 15, 10, 7.5, 6, 5, 4, 3 or 2 mm from the inner surface of the light transparent member. The predetermined point can be 2-5mm from the inner surface of the light transparent member. The predetermined point can be at a depth equal to or less than 20, 15, 10, 7.5, 6, 5, 4, 3 or 2 mm in the well fluid. The predetermined point can be located adjacent to or near the center of the light transparent member, which can be a generally circular cross section, a flat circular or disc shape, or a chamfered or beveled edge.

The chamfered or beveled edge can be an edge proximal to the well fluid. The chamfered edge can be adapted to increase turbulence at or near the surface of the light transparent member.

The light transparent member can have a thickness of about 3-7, 4-6 or 5 mm and have a hardness of at least 7, 8, 9 or 10 on Mohs scale of hardness. The light transparent member can have a compressive strength of at least 0.5, 0.75, 1, 1.25, 1.5, 1.75 or 2GPa.

The light transparent member can be inert and/or otherwise resistant to corrosion. The light transparent member can have a thermal conductivity at 100° C. of no more than 5, 10, 15, 20 or 25 W/m.K. The light transparent member may have a thermal expansion co-efficient (at 20 to 50° C.) of no more than 4.5×10−6/° C., 5×10−6/° C., 5.25×10−6/° C., 5.75×10−6/° C. or 5.8×10−6/° C.

The light transparent member can be of sapphire or sapphire glass and may also be formed of quartz or some other material with equivalent physical characteristics such as optical and strength properties. The light transparent member is adapted to form a seal that prevents passage of the well fluid and can form a window such as an optical window or lens. The lens can have at least one curved surface.

The sensor assembly may be a housing for locating the first light source, the first light detector, and the light transparent member in position relative to each other. The housing may be adapted to be attached to a section of a pipeline for the well fluid wherein the pipeline defines a flow direction for the well fluid. The section of a pipeline may be located above ground and/or may be located adjacent a well head.

The housing can be adapted to be attached to the pipeline by being integrated into or forming a section of the pipeline. In this regard, the housing may further include a pipe section adapted to interface with and form a section of the pipeline. The pipe section defines two interfaces or flanges to enable the housing to be removably attached to form part of the pipeline.

The flanges define a mounting arrangement for attaching the housing to the pipeline. The housing may also be attached to the pipeline by forming an aperture in the section of the pipeline and affixed the housing to the section adjacent to the aperture. The housing can be adapted to be attached to the pipeline so that the first light enters the well fluid from a point to the side or located laterally relative to the flow direction of the well fluid. Thus, the sensor assembly may be adapted to be side mounted to the pipeline.

The sensor assembly can further include a seal that prevents the well fluid from passing the light transparent member. The seal may be varied but includes a deformable seal located at or near the outer edge of the light transparent member.

The physical characteristics of the well fluid may impact the detection of the fluorophore. Thus, the sensor assembly may further include:
(a) a second light source for emitting a second light into the well fluid; and
(b) a second light detector for detecting a light emitted from the well material in response to the second light;
(c) wherein the second light source and second light detector form a second optical arrangement for measuring a physical characteristic of the well fluid.

The physical characteristic mentioned above can be turbidity. The second light source mentioned above may be, but is not required to be, a light emitting diode (LED) and can have a leading end facing the light transparent member and a trailing end facing the opposite direction.

The second light source may emit the second light to the predetermined point or to a further predetermined point at the same or similar depth in the well fluid. The second light source can be adapted to emit infrared light. The second light source may be oriented such that, when in use, its central axis defines an acute angle with the outer surface of the light transparent member. Preferably, the acute angle is about 40-80, 50-70, 55-65 or 60°. The second light source may further include a third filter that only allows the passage of light of a predetermined wavelength or wavelength range.

The predetermined wavelength or wavelength range is 750-950 nm, 775-925 nm, 800-900 nm or 825-875 nm.

The second light can be infrared light and/or operate at a wavelength of 750-950 nm, 775-925 nm, 800-900 nm or 825-875 nm. The second light detector may be a photodiode, a phototransistor, or a photoresistor. The second light detector can have a leading end facing towards the light transparent member and a trailing end facing in the opposite direction. The second light detector can be oriented such that, when in use, its central axis defines an acute angle with the outer surface of the light transparent member and the acute angle can be about 40-80, 50-70, 55-65 or 60°.

Preferably, the second light detector is oriented such that its central axis and the central axis of the second light source converge in a direction towards the predetermined point to define a second angle of convergence.

Preferably, the second angle of convergence is an acute angle. Even more preferably, the second angle of convergence is about 40-80, 50-70, 55-65 or 60°.

The second light detector may further includes a fourth filter that only allows the passage of light of a predetermined wavelength or wavelength range. Preferably, the predetermined wavelength or wavelength range is 750-900 nm, 775-875 nm or 800-850 nm. The second light detector can be located in a position opposed to the second light source such that the second light source and the second light detector are opposed to each other.

The light emitted from the well fluid in response to the second light is a wavelength or wavelength range of 750-900 nm, 775-875 nm or 800-850 nm.

The second light source and second light detector can form a second optical arrangement for measuring a physical characteristic of the well fluid.

The sensor assembly may form part of a sensor unit. Thus, according to a second aspect, the present invention provides a sensor unit that includes:
(a) a sensor assembly according to a first aspect of the present invention; and
(b) a signal processor for processing a signal generated by the sensor assembly.

The sensor assembly can be running continually and in real time. The signal can be derived from the first light detector and/or the second light detector. The signal processor can be adapted to convert an analogue signal to a digital signal or vice versa. The signal processor can be adapted to amplify the signal to produce an amplified signal. The signal processor can be adapted to sample the signal and/or the amplified signal.

The signal processor is capable of performing one or more of the following:
(a) applies signals from the first and/or the second light detector to a mathematical model which counteracts the effects of process fluid conditions, such as turbidity and/or internal pipe reflections; and
(b) processes the signal to account for the temperature of the sensor unit.

The signal processor can be adapted to produce an output signal with a reduced noise floor, thus improving detectability of fluorophore concentrations or otherwise improving the signal to noise ratio of a signal from the first and/or second light detector.

The sensor unit can be sealed to define an air pocket therein.

The sensor assembly according to the first aspect of the present invention or the sensor unit according to the second aspect of the present invention may form part of an insert for a pipeline. Thus, according to a third aspect, the present invention provides an insert for a pipeline that includes:
(a) a sensor assembly according to a first aspect of the present invention or a sensor unit according to a second aspect of the present invention; and
(b) a well fluid conduit defining an inlet and an outlet.

The insert can be adapted to form a fluid tight seal with the pipeline via a sealing structure.

The sealing structure includes at least one pipe flange. The sealing structure can include two pipe flanges, one located at the inlet and one located at the outlet. The sealing structure can also include a threaded member adapted to threadingly engage with a compatible threaded member on the pipeline, such as a collar.

The well fluid conduit can include a section of pipe between the inlet and the outlet. The well fluid conduit can be adapted to modify the flow of well fluid passing therethrough, increase and/or decrease the flow rate of well fluid passing therethrough relative to the flow rate in the pipeline, increase the turbulence of well fluid passing therethrough, and/or increase the turbulence of the well fluid passing therethrough at or near the sensor assembly or the sensor unit.

The well fluid conduit may have an internal profile that is shaped to modify the flow of well fluid passing therethrough. The well fluid conduit can define a first section with a first cross-section area, a second section with a second cross section area and a third section with a third cross sectional area. The first cross sectional area can be smaller than the second cross sectional area. The third cross sectional area can be smaller than the second cross sectional area. The first section and the third section can be located on either side of the second section. The first section and/or the third section can define a tapered section. The first, second, and third sections can be formed into a section of pipe.

The insert for a pipeline may form part of a pipeline and thus the present invention also provides a pipeline including the insert for a pipeline as described herein.

One or more of the aspects of the invention described above can be used in a method to detect wear in a well component. Thus, according to a fourth aspect of the invention, the present invention provides a method of detecting wear of a well component in a well, the method including the steps of:
(a) incorporating a well component, including a fluorophore, in the well; and
(b) assaying the well fluid for the fluorophore;
   wherein the presence of the fluorophore in the well fluid is indicative of the wear.

Step (b) can include the use of a sensor assembly as described herein with reference to the first aspect of the invention.

The well component can be selected from the group including: a rod guide such as a sucker rod guide, a well liner or tube, a rod such as a sucker rod and a fitting or coupling for any of the aforementioned well components. The well component can be a rod guide.

The fluorophore can be included in a substrate that forms part of the well component. The substrate can be less resistant to wear than the remainder of the well component. The fluorophore can be included in the substrate in a retaining means for the fluorophore. The retaining means may include a cavity or void.

The shape and configuration of the cavity or void and the number thereof depend on the nature of the performance monitor component. In some embodiments a single cavity or void is provided. In other configurations, a plurality of cavities or voids are provided.

When a plurality of cavities or voids is provided, they may be provided at the same depth within the well component or at different depths therein. In this regard, different levels of wear can be identified by including a plurality of cavities or voids at different depths within the well component.

The retaining means may also include a separate component in the form of a receptacle that defines the cavity or void. The receptacle can be embedded in the well component and/or can be formed from a material that is less wear resistant than the well component. The receptacle may include a removable cap and a body. In this regard, the removable cap can be removed to enable the fluorophore to be inserted into the body of the receptacle.

The removable cap can be adapted to form a friction fit with the body of the receptacle. The removable cap can be formed from a material that is less wear resistant than the well component.

The well component can include a plurality of receptacles.

The physical characteristics of the well fluid may impact the detection of the fluorophore. Thus, the method of detecting wear may further include the step of:
(c) measuring a physical characteristic of the well fluid.

The measuring step includes quantifying the physical characteristic. The physical characteristic can be turbidity. The measure of the physical characteristic can be used to inform the step of assaying the well fluid for the fluorophore. For example, the measure of the physical characteristic may be used to process a signal based on the assay for the fluorophore. Even more preferably, the measure of the physical characteristic may be used to amplify the signal relative to noise in the signal.

One or more of the aspects of the invention described above can be included in a wear detection system. Thus, according to a fifth aspect of the invention, the present invention provides a wear detection system including:
(a) a well component including a fluorophore; and
(b) a sensor assembly, a sensor unit and/or an insert for a pipeline as described herein.

### Advantages

Whilst not limited to the following, applicant believes the present invention and/or preferred embodiments thereof have a number of advantages including one or more of the following:
(i) the invention is capable detection of fluorophore within highly turbid well fluids;
(ii) fluorophore detection provides wear information on downhole well components, which may be used to provide advance warning before damage is caused. Maintenance can be scheduled in advance, minimizing well down-time and costs;
(iii) the invention relies on non-intrusive optical methods to detect fluorophore and process
(iv) the invention can be installed as an in-line insert for a pipeline, minimizing required modifications to existing pipelines;
(v) the invention can be designed to be intrinsically safe; and/or
(vi) the invention allows for the use of highly flexible communication interface(s) which can be used by external systems to retrieve live and/or historic data as well as configure the sensor assembly/unit.

### General

Those skilled in the art will appreciate that the invention described herein is susceptible to variations and modifications other than those specifically described. The invention includes all such variation and modifications. The invention also includes all of the steps and features referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. None of the cited material or the information contained in that material should, however be understood to be common general knowledge.

The present invention is not to be limited in scope by any of the specific embodiments described herein. These embodiments are intended for the purpose of exemplification only. Functionally equivalent products and methods are clearly within the scope of the invention as described herein.

The invention described herein may include one or more range of values (e.g., size etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all technical terms used herein have the same meaning as commonly understood to one of ordinary skill in the art to which the invention belongs.

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. In the various Figures the same reference numerals have been used to identify similar elements.

### DETAILED DESCRIPTION OF THE PREFERRED EMBODIMENTS

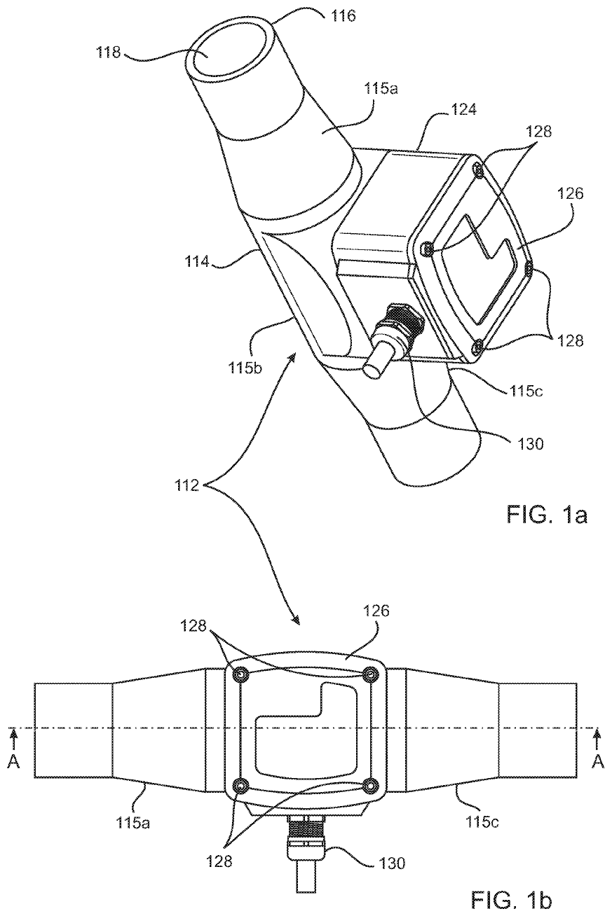

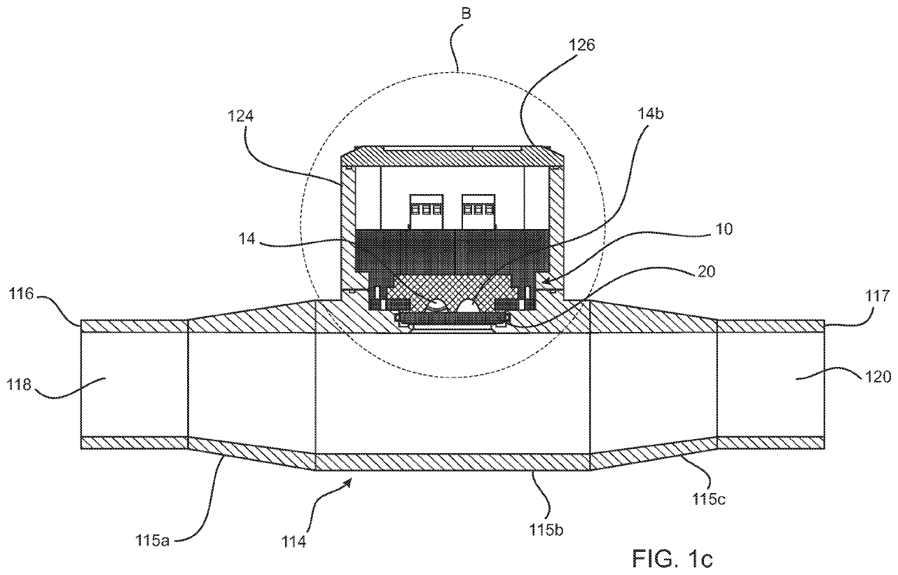

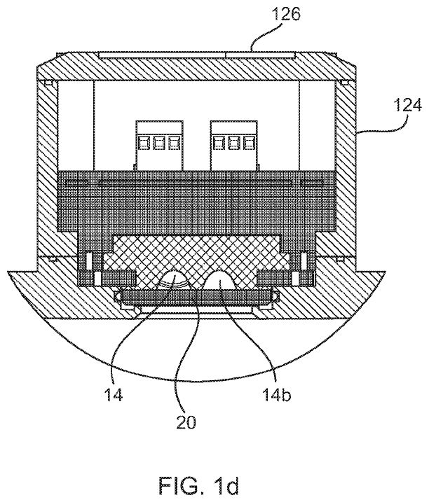

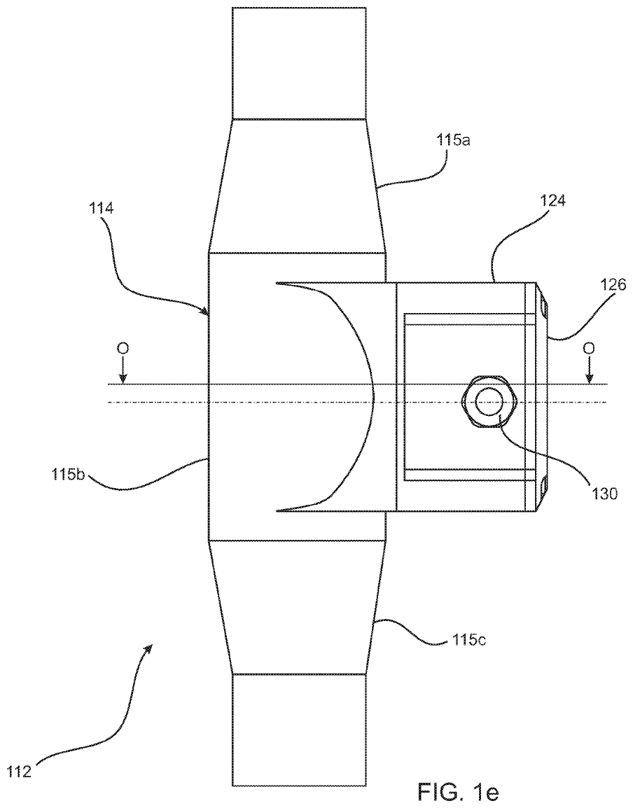

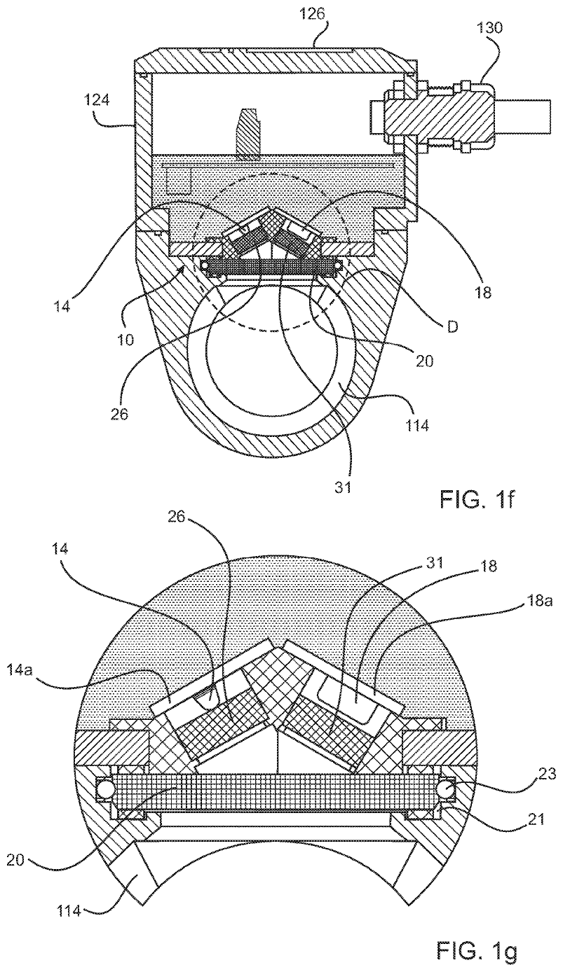

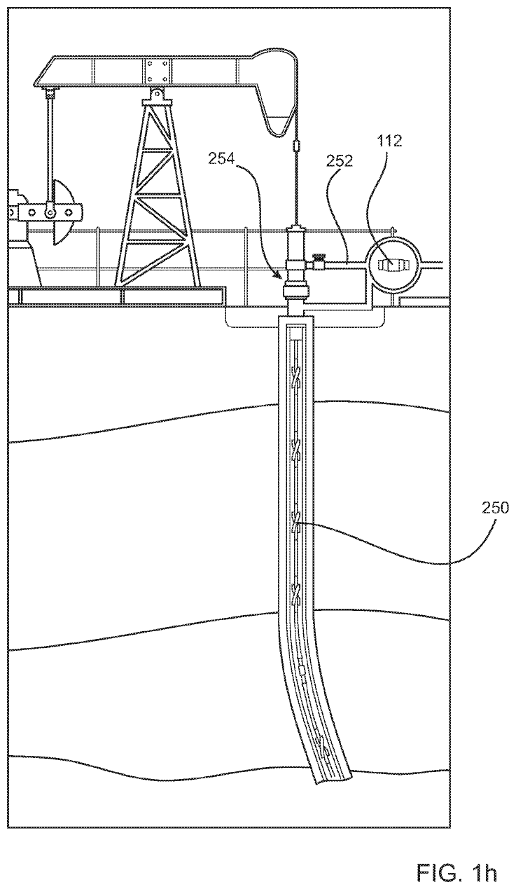

A sensor assembly according to an embodiment of the first aspect of the present invention, and generally indicated by the numeral 10, is depicted in FIG. 1a-g as part of an insert for a pipeline 112 according to an embodiment of the third aspect of the present invention. One example of where the insert 112 can be installed as part of a well is shown in the schematic diagram in FIG. 1h.

The sensor assembly 10 includes a first optical arrangement includes a first light source in the form of first LED 14 and first light detector in the form of first photodiode 18 for detecting a fluorophore and a second optical arrangement includes a second light source 14b and second light detector 18b for measuring turbidity. The sensor assembly 10 is described in more detail later herein.

The insert 112 includes a well fluid conduit in the form of a section of pipe 114 that includes an inlet 118 and an outlet 120 at either end. The insert 112 is adapted to be integrated into a pipeline via a sealing means in the form of a first friction fitting 116 located adjacent the inlet 118, and a second friction fitting 117 located adjacent the outlet 120. The friction fittings 116, 117 are adapted to allow for the insert 112 to be included in a pipeline such as well pipeline using a glue, plastic cement or some other adherent to attach the insert 112 to the pipeline. The friction fittings 116, 117 may also incorporate a threaded section or a threaded collar that is compatible with a threaded section in the pipeline. In another embodiment the insert is included in a pipeline via the use of a first pipe flange 216 and a second pipe flange 217 (best shown in FIG. 5) that each include a series of holes 219 to allow the insert 112 to be fixed in the pipeline using a set of threaded fixing means such as bolts or the like.

The section of pipe 114 is adapted to modify the flow of well fluid passing therethrough during use. In this regard, the pipe 114 includes a first section 115a adjacent to the inlet 118 with an internal cross-sectional area that tapers out in the direction of the flow of well fluid 122, during use, a second section 115b that has a consistent internal cross-sectional area, and a third section 115c adjacent to the outlet 120 with an internal cross-sectional area that tapers out in the direction of the flow of well fluid, during use, as shown by arrow 122. The arrangement of the sections 115a-c acts to:
(i) avoid constricting the flow rate of the well fluid as it enters and passes therethrough that would induce (unwanted) backpressure into the system;
(ii) provide a “viewing” area for the sensor to “see” into the well fluid that is flat to allow for controlled or otherwise predictable refraction of light; and
(iii) provide a gradual/steady transition for the flow of the well fluid to minimise changes in velocity and pressure in the well fluid.

The above in combination with the chamfered edge 21 on the sapphire glass disc 20, that increases the turbulence of well fluid passing thereby provides for optimal performance of the sensor assembly 10, in terms of its ability to accurately detect the fluorophore in the well fluid.

The sensor assembly 10 is mounted to the insert 112 via the second section 115b so that when the insert 112 is attached to a pipeline and properly oriented, the sensor 10 is directed towards the inside of the insert 112 at a point adjacent to the second section 115b.

The sensor assembly 10 is supported in a housing in the form of a casing 124 that includes a front facing panel 126 section that is removably held in place by four threaded engagement members in the form of bolts 128. Power is delivered to the sensor assembly 10 and data is delivered and retrieved from the sensor via cabling that enters the casing 124 from the bottom surface thereof via a cable fitting 130.

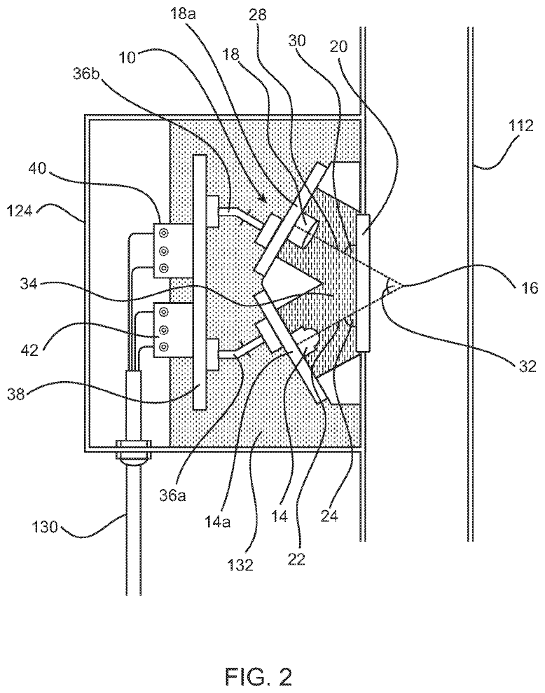

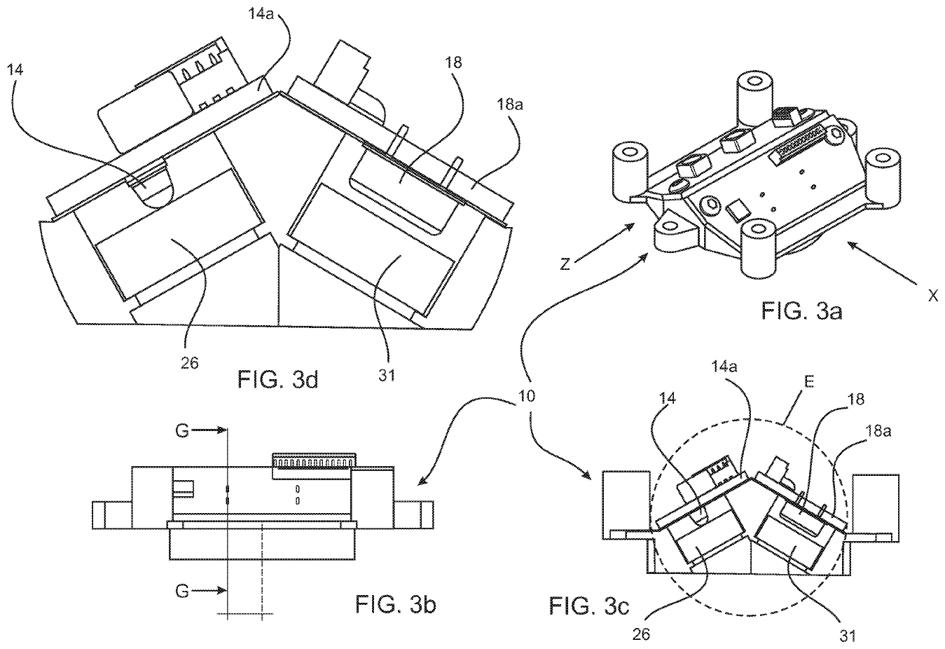

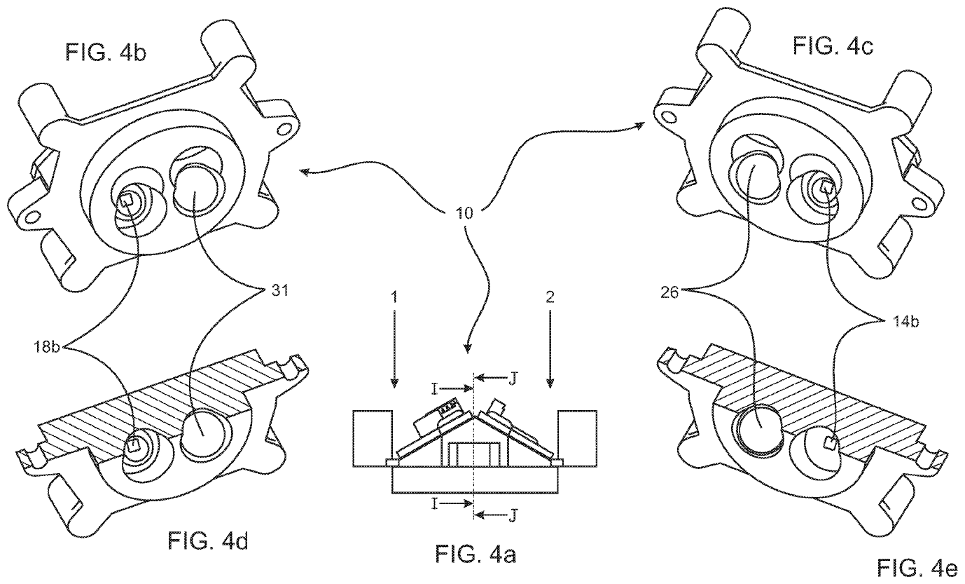

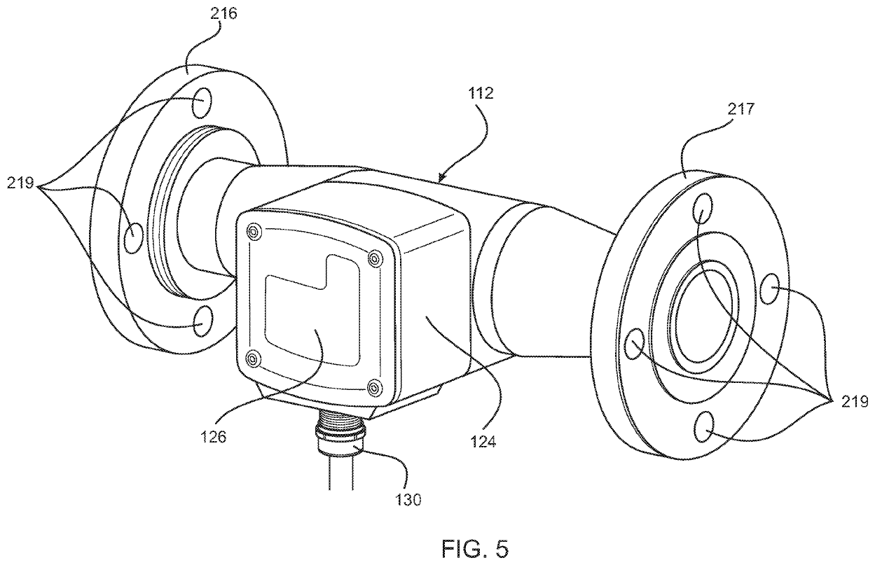

The sensor assembly 10, which will now be described in more detail with reference to FIG. 2, is for detecting a fluorophore, in the form of PTSA, in the well fluid. The sensor assembly 10 includes a first light source in the form of a first LED 14 for emitting a first light with a wavelength of about 367 nm. The first light passes through a light transparent member, in the form of 5 mm thick sapphire glass disc 20, to a predetermined point 16 located inside a section of pipe 112 for carrying well fluid and about 4 mm from the inner surface of the sapphire glass disc 20. When the first light contacts PTSA in the well fluid, light emitted from the PTSA in response to the first light, with a wavelength (about 375 nm-475 nm) passes through the second band pass filter 31 that only allows the passage of light of about 425 nm (range is 400-425 nm) that is detected by a first light detector in the form of a first photodiode 18.

The first LED 14 is oriented such that its central axis 22 defines an acute angle 24 of about 60° with the outer surface of the sapphire glass disc 20. The first LED 14 further includes a first filter in the form of a first band pass filter 26 that only allows the passage of light of about 350 nm.

The sapphire glass disc 20 includes a chamfered or beveled edge 21 towards its inner surface which is proximal to the section of pipe 112 to assist with locating the sapphire glass disc 20, forming a fluid tight seal with the section of pipe 112 and creating turbulence at or near the surface of the sapphire glass disc 20. This fluid tight seal includes a sealing member in the form of a resiliently flexible O-ring 23, that extends radially around the outer edge of the sapphire glass disc 20, and a retainer (not shown) that can be adjusted to clamp the sapphire glass disc 20 against the section of pipe 112.

The first photodiode 18 is oriented such that its central axis 28 defines an acute angle 30 of about 60° with the outer surface of the sapphire glass disc 20. The first photodiode 18 further includes a second filter in the form of a second band pass filter 31 that only allows the passage of light of about 400-425 nm (425 nm peak).

The first LED 14 and the first photodiode 18 form a first optical arrangement and are oriented such that their respective central axes 22, 28 converge in a direction towards the predetermined point 16 to define an angle of convergence 32 of about 60°.

A second optical arrangement is included in the sensor assembly 10. It includes a second light source in the form of a second LED 14b for emitting a second light with a peak wavelength of about 850 nm. The second light passes through the sapphire glass disc 20, to the predetermined point 16. When the second light contacts suspended material in the well fluid, light reflected in response to the second light with a peak wavelength of about 850 nm is detected by a second light detector in the form of a second photodiode 18b.

The second LED 14b is oriented such that its central axis defines an acute angle of about 60° with the outer surface of the sapphire glass disc 20.

The second photodiode 18b is oriented such that its central axis defines an acute angle of about 60° with the outer surface of the sapphire glass disc 20.

The second LED 14b and the second photodiode 18b form a second optical arrangement and are oriented such that their respective central axes converge in a direction towards the predetermined point 16 to define an angle of convergence of about 60°.

The first and second optical arrangements are located in a hermetically sealed air pocket 34 and the sensor assembly 10 further includes a range of circuitry located outside of the air pocket 34. In particular, the first LED 14, the second LED 14b and the first photodiode 18 and the second photodiode 18b are mounted to PCBs 14a, 18a respectively which are connected via connectors 36a, 36b to a main PCB 38. PCB 14a includes high efficiency LED driver circuits. PCB 18a includes precision analog to digital converters which obtain samples from the photodiode circuits. The main PCB 38 includes a processor for the signals generated by the photodiodes and also manages the transfer of incoming and outgoing data and communications. A further PCB (not shown) manages the safety protocols of the sensor assembly 10 including ensuring compliance with intrinsic safety requirements applicable in the oil and gas industry. This further PCB mounts directly to the main PCB 38. Power and data transmission occurs via wired connections that run from the terminal blocks 40, 42 to the exterior of the sensor assembly 10 via a cable that passes through cable fitting 130 that passes through a suitably formed and sealed aperture in the casing 124.

For safety reasons, the majority of the components, and in particular the PCBs, described above are encapsulated in a suitable material in the form of epoxy resin 132 to prevent them from becoming an ignition source.

When used in a pipeline the sensor assembly is able to detect the presence of PTSA in the well fluid. In this regard, when the sensor forms part of a wear detection system (best shown in FIG. 1h), it can detect PTSA released, due to wear, from a well component in the form of a sucker rod guide 250 into the well fluid. Presence of the PTSA is detected by the sensor located in the insert 112 that is located in line with the well pipeline 252 adjacent to the well head 254, and in particular the first optical arrangement, that generates a signal that can be processed and used by an operator to better manage the operation of the well. In this regard, presence of the PTSA can indicate to an operator that a particular well component, such as a sucker rod guide, requires servicing or replacement to ensure acceptable performance of the sucker rod guide and operation of the well.

The second optical arrangement measures the turbidity of the well fluid and this parameter can be useful in terms of the functioning of the sensor. In this regard, turbidity of the well fluid may impact on the detection of the PTSA and increased turbidity may result in a decrease in the signal to noise ration of the PTSA. Thus, when processing the signal generated by the sensor based on detection of the PTSA, the turbidity of the well fluid detected by the second optical arrangement may be accounted for and adjustments made to avoid false positives or negatives from the first optical arrangement.

The claims appended hereto are meant to cover all modifications and changes within the scope and spirit of the present invention.

1-50. (canceled)

51. A sensor assembly for detecting a fluorophore in a well fluid, the sensor assembly comprising:
(a) a first light source for emitting a first light to excite the fluorophore at a predetermined point in the well fluid;
(b) a first light detector for detecting a light emitted from the fluorophore in response to the first light; and
(c) a light transparent member located between the well fluid and the first light source and first light detector and defining an outer surface and an inner surface in fluid communication with the well fluid.

52. A sensor assembly according to claim 51 wherein the fluorophore has an excitation wavelength of 325-425 nm.

53. A sensor assembly according to claim 51 wherein the fluorophore has an excitation wavelength of about 350 nm.

54. A sensor assembly according to claim 51 wherein the fluorophore is 1,3,6,8-pyrene tetrasulfonic acid tetrasodium salt (PTSA).

55. A sensor assembly according to claim 51 wherein the well fluid comprises a turbidity of at least 15000 NTU.

56. A sensor assembly according to claim 51 wherein the first light source is oriented such that, when in use, it's central axis defines an acute angle with the outer surface of the light transparent member.

57. A sensor assembly according to claim 51 wherein the first light source further comprises a first filter that only allows the passage of light of a predetermined wavelength or wavelength range.

58. A sensor assembly according to claim 57 wherein the predetermined wavelength or wavelength range is 320-380 nm.

59. A sensor assembly according to claim 51 wherein the first light detector is oriented such that, when in use, it's central axis defines an acute angle with the outer surface of the light transparent member.

60. A sensor assembly according to claim 51 wherein the first light detector is oriented such that its central axis and the central axis of the first light source converge in a direction towards the predetermined point to define an angle of convergence.

61. A sensor assembly according to claim 60 wherein the angle of convergence is an acute angle.

62. A sensor assembly according to claim 61 wherein the first light detector further comprises a second filter that only allows the passage of light of a predetermined wavelength or wavelength range.

63. A sensor assembly according to claim 62 wherein the predetermined wavelength or wavelength range comprises 390-460 nm.

64. A sensor assembly according to claim 51 wherein the first light detector is located in a position opposed to the first light source.

65. A sensor assembly according to claim 51 wherein the predetermined point is located proximal to the inner surface of the light transparent member.

66. A sensor assembly according to claim 51 wherein the light transparent member comprises a chamfered or bevelled edge proximal to the well fluid.

67. A sensor assembly according to claim 51 wherein the light transparent member comprises a hardness of at least 7, 8, 9 or 10 on Mohs scale of hardness.

68. A sensor assembly according to claim 51 wherein the light transparent member comprises sapphire or sapphire glass.

69. A sensor assembly according to claim 51 further comprising a housing for locating the first light source, the first light detector and the light transparent member in position relative to each other.

70. A sensor assembly according to claim 51 further comprising:
(a) a second light source for emitting a second light into the well fluid; and
(b) a second light detector for detecting a light emitted from the well material in response to the second light;
wherein the second light source and second light detector form a second optical arrangement for measuring a physical characteristic of the well fluid.
